(19) United States
(12) Patent Application Publication
(10) Pub. No.: US 2022/0145382 A1
(43) Pub. Date: May 12, 2022

(54) Precise and Programmable DNA Nicking System and Methods

(71) Applicant: Genvida Technology Company Limited, Tortola (VG)

(72) Inventors: Ka Wai WONG, Hong Kong (CN); Chi Yip HO, Toronto (CA); Daniel Wai-Cheong SO, Palo Alto, CA (US)

(21) Appl. No.: 17/383,633

(22) Filed: Jul. 23, 2021

Related U.S. Application Data
(60) Provisional application No. 63/111,124, filed on Nov. 9, 2020.

(51) Int. Cl. C12Q 1/6869; G01N 33/487

(52) U.S. Cl. CPC C12Q 1/6869; G01N 33/48721

(57) ABSTRACT

Nicking molecules nick or cleave a polynucleotide molecule translocating through a real-time single-base-read nanopore sequencing device that achieves real-time single-base-read sequencing by probing individual bases of the polynucleotide molecule. The polynucleotide molecule is guided to enter and translocate through a nanopore of the nanopore sequencing device. A target sequence is determined for a nick or cleave, and the polynucleotide molecule is sequenced by the nanopore sequencing device. After reading the target sequence, an external excitation is applied to trigger one or more nicking molecules and thereby nick or cleave the polynucleotide at a location adjacent to the one or more nicking molecules. In the case of a requirement to further nick or cleave the same target sequence, the process is continued, and in the case of a requirement to nick or cleave another target sequence, another target sequence is determined.

### BACKGROUND

### Technical Field

The disclosed technology pertains to a method and apparatus for nicking polynucleotides, and more specifically, for precise and programmable polynucleotide nicking through a synergistic combination of real-time sequencing at single-base resolution (hereafter “single-base-read”), and a molecular nicking action.

### Background Art

Nicking or cleaving is a pivotal step for most nucleic acid manipulation in vivo and in vitro in genomics and bioengineering, as evinced by the plethora of molecular motors working vigorously all the time along the chromosomes of living cells. Endonucleases are enzymes that cleave a polynucleotide chain by separating nucleotides of the polynucleotides. Restriction endonucleases (REs) are among important molecular tools that manipulate primarily double stranded DNA by generating nicks or cleavages. Since the discovery of the DNA double-helix structure by Watson and Crick in 1953, numerous types of REs have been discovered. REs can be classified depending on their structure, specific recognition sequences, catalytic activity and nicking or cleaving locations and molecules. Different REs are used in nucleic acid manipulation, analysis and sample preparation in applications including cloning, study of polymorphism, methylation profiling, gene expression analysis, and optimization of high-throughput DNA sequencing.

REs consist of a recognition domain and a cleavage domain. The former recognizes and binds to a specific sequence of nucleotides, referred to as a recognition sequence. A recognition sequence usually has between 4 and 8 bases. Upon binding to this recognition sequence with the recognition domain, different nicking or cleaving actions are exerted on, near or away from the recognition sequence by the cleaving domain, depending on the type of RE used. Since the binding action of REs is sequence-specific, the nicking or cleavage location is dependent on the presence of the recognition sequence (regardless of where precisely this location may be after such sequence recognition). Such sequence-specificity limits the number of sites for nicking or cleaving along the DNA. The DNA will not be nicked at just any point after a particular sequencing of interest because the nicking action is strictly coupled to the specific sequence recognition by the RE.

If one wants to nick or cleave at different sites along the same DNA, i.e., sites after different recognition sequences, multiple REs are required to recognize each recognition sequence of interest. The use of multiple REs can adversely complicate or interfere with both the accuracy and precision of the enzymes. For instance, the RE BamHI is well known to exhibit nonspecific actions in suboptimal buffer conditions. The situation can be even worse if using different REs that require incompatibly different conditions to stay active.

If the nicking or cleaving action could be achieved at will (i.e., at or near any desired polynucleotide sequence), without limitation to any specific recognition sequence, the scope of nucleic acid engineering and manipulation could be greatly broadened. Many more novel applications could be explored and developed. For example, fabricating DNA punch cards through topological modifications could be realized for DNA-based data storage.

To detect a desired sequence of a polynucleotide requires accurate sequencing. One class of technologies used for sequencing polynucleotides are nanopore-based sequencing technologies. Nanopores can be broadly categorized into two types: biological and solid-state. Both of types have been used and proved applicable for sequencing. Biological nanopores, i.e., known as transmembrane protein channels, are usually inserted into a planar substrate such as lipid bilayers, or liposomes to form the sensing platform. Examples are α-Hemolysin, MspA and Bacteriophage phi29. Solid-state nanopores refer to those made of inorganic materials such as oxides (e.g. Al₂O₃, SiO₂), nitrides (e.g. Si₃N₄), 2D materials (e.g. graphene, MoS₂), polymers. Solid-state nanopores are usually fabricated by physical processes, for instance, ion or electron beam bombardment, electrochemical etching, ion-tracked etching.

It has been demonstrated that nanopore can be used for sequencing. In 1996, Brandon et. al. firstly reported the use of nanopore for biological studies (Kasianowicz J. J., Brandin E., Branton D., Deamer D. W., “Characterization of individual polynucleotide molecules using a membrane channel”, Proc. Natl. Acad. Sci. USA 93, 13770-13773, 1996). More information related to DNA sequencing using nanopore can be found in R. M. Venkatesan, R. Bashir, “Nanopore sensors for nucleic acid analysis”, Nat. Nanotechnol. 6, 615-624, 2011, and Miles B. N., Ivanov A. P., Wilson K. A., Do\check{g}an F., Japrung D., Edel J. B., “Single molecule sensing with solid-state nanopores: novel materials, methods, and applications”, Chem. Soc. Rev. 42, 15-28, 2013. Briefly, a nanopore is a hole passing through a membrane or substrate, the hole having a nanoscale dimension (i.e., diameter between 1 nm to 999 nm). To be used in sequencing, the nanopore has a diameter that allows passage of a single-stranded (s.s.) DNA or RNA molecule (e.g., with an inner diameter between 1 and 20 nm, or 2 and 10 nm or 2 and 5 nm) from one side of the membrane or substrate. As the DNA or RNA molecule passes through the nanopore, a property directly or indirectly derived from individual bases translocating through the nanopore are distinctively probed and measured, rendering individual bases identifiable and sequenceable.

One example of nanopore-based sequencing is sequencing by monitoring the current blockage when a DNA or RNA molecule passes through a nanopore that separates two compartments (cis and trans). An initial current is created through the nanopore by an applied voltage across a membrane or structure containing the nanopore. The structure can be a synthetic or natural structure capable of being traversed by the nanopore. When a nucleotide passes through and (entirely or partially) blocks the nanopore, it excludes a certain volume of ions in the buffer solution and causes a reduction of current across the nanopore (blockage current). Since the volume of ions excluded, and thus the blockage current, is related to physical and chemical characteristics of each individual nucleotide, each nucleotide passing through can be identified based on that blockage current, and the DNA can be sequenced (see, e.g., FIG. 1A).

Another example of nanopore-based sequencing is sequencing by monitoring the in-plane tunneling current across each nucleotide passing through the nanopore. When each nucleotide passes through the nanopore, it can be probed separately by surrounding in-plane electrodes embedded within the nanopore through in-plane tunneling current measurement (see, e.g., FIG. 1B). Since each nucleotide has its own characteristic electronic structure and thus conductivity, the change in tunneling current can distinguish the nucleotide passing through, and sequencing is therefore achieved.

Thus, a method and apparatus for precise and programmable polynucleotide nicking based on a nanopore-based real-time single-base-read sequencing device functionalized by one or more nicking molecules solving the aforementioned problems is desired.

### SUMMARY

Polynucleotide molecule (DNA, RNA or any synthetic or natural nucleic acid) nicking or cleaving is achieved by providing one or more nicking molecules coupled to a single-base-read nanopore sequencing device to perform a nicking or cleavage action on a DNA molecule passing through the single-base-read nanopore sequencing device. The single-base-read nanopore sequencing device achieves real-time single-base-read sequencing by probing an individual base of the polynucleotide molecule passing through it. The one or more nicking molecules with the single-base-read nanopore sequencing permits nicking or cleaving of the polynucleotide molecule without restriction of any particular sequence for recognition and/or binding.

Coupling or grafting of nicking molecules to a nanopore can be achieved based on any well-established or otherwise suitable conjugation chemistry. Coupling or grafting could comprise physisorption, chemisorption or covalent bonding of the nicking molecule to a location at a defined location relative to, such as directly adjacent to, the nanopore of the single-base-read nanopore sequencing device. For example, in an embodiment, the nicking molecule has a first functional group and can be covalently linked to a designated location near the nanopore functionalized with a second functional group, wherein the first and second functional groups couple directly or with the help of a mediating agent. Non-limiting examples of functional group pairs that couple are carboxylic acid (—COOH) with amine (—NH₂), chloromethyl (—CH₂Cl) with amine (—NH₂), sulphide (—S) with gold (Au), and sulphide (—S) with sulphide (—S), each of which have been extensively studied and applied to functional coupling of biomolecules together or to desired substrates or targets. Considering the coupling pair of carboxylic acid (—COOH) with amine (—NH₂) as an example, to graft a nicking molecule to the nanopore, the designated point of attachment on the nanopore is firstly functionalized with one of the chemical groups in the chosen coupling pair, e.g., —COOH, by either chemical or physical treatment. The nicking molecule is similarly functionalized with the other member of the coupling pair, i.e., —NH₂, if needed. The NH₂-modified nicking molecule can then be grafted on to the COOH-modified nanopore at the designated region in appropriate conditions, which would be well known and conventional to one skilled in the art.

In one embodiment, precise and programmable DNA nicking is performed by guiding a polynucleotide molecule to a nanopore of the real-time single-base-read nanopore sequencing device and guiding the polynucleotide molecule to enter and translocate through the nanopore. A first target sequence is determined for a nick or cleave, for example by a user or an algorithm implemented by a computer or processor, and the polynucleotide molecule is sequenced by the real-time single-base-read nanopore sequencing device. After reading the first target sequence by the real-time single-base-read nanopore sequencing device, an external excitation is applied to trigger the one or more nicking molecules to nick or cleave an adjacent nucleotide base of the polynucleotide molecule. In the case of a requirement to further nick or cleave the polynucleotide molecule following further instances of the first target sequence in the polynucleotide molecule, the process is continued. In the case of a requirement to nick or cleave following a second target sequence, said second target sequence becomes a new target sequence written to the real-time single-base-read nanopore sequencing device. An external excitation can be used to trigger the one or more nicking molecules to nick the adjacent nucleotide base of the polynucleotide molecule, and the process can be repeated until completion of all nicks or cleaves.

In another aspect, polynucleotide based information storage is achieved through generation of nicks or cleavages as registers on a polynucleotide substrate by one or more nicking molecules. A predetermined sequence of one or more nucleotide bases of the polynucleotide substrate as a nicking or cleaving recognition sequence is selected. The DNA substrate is sequenced in real-time and, in order to write a register “1”, a nick is generated after reading the nicking or cleaving recognition sequence by triggering an external excitation to activate the one or more nicking or cleaving molecules. To write a register “0”, a nicking or cleaving action is skipped even after reading the nicking or cleaving recognition sequence. The process is continued and repeated until the desired sequence of registers are made.

### BRIEF DESCRIPTION OF THE DRAWINGS

FIG. 1A is a schematic drawing showing an exemplary nanopore device for DNA sequencing by ion blockage current measurement.

FIG. 1B is a schematic drawing showing an exemplary nanopore device for DNA sequencing by in-plane electrical signal measurement.

FIG. 2A is a schematic drawing illustrating an exemplary setup for the disclosed technology consisting of nicking molecules grafted at the opening of a nanopore and a single-base-read nanopore sequencing system for precise and programmable DNA nicking immediately after the desirable DNA sequence read.

FIG. 2B is a schematic drawing illustrating an exemplary setup for the disclosed technology consisting of nicking molecules grafted at a predetermined distance above the opening of a nanopore and a single-base-read nanopore sequencing system for precise and programmable DNA nicking at a designated site away from the desirable DNA sequence read.

FIG. 3 is a diagram illustrating the implementation of precise nicking using single-base-read nanopore sequencing device of the disclosed technology.

FIG. 4 is a flow chart illustrating the method of the disclosed technology.

FIG. 5 is a schematic diagram illustrating a random access memory employing a nucleic acid.

FIG. 6A is a flow diagram showing an example of an application in synthetic biology using natural DNA instead of conventionally chemical synthesized DNA oligonucleotides.

FIG. 6B is a diagram showing an example of a sequence of removal of two introns from the yeast MATa1 genomic DNA (in two iterations of single intron removal) using an embodiment of the disclosed technology.

### DETAILED DESCRIPTION

### Overview

The disclosed technology is related to a system and a method for precise and programmable polynucleotide nicking or cleaving based on a hybrid structure consisting of nicking molecules and a single-base-read nanopore sequencing device. The technique can be implemented without dependence on a specific polynucleotide sequence for recognition, such as those required for nicking or cleaving by REs following bonding with their recognition domains.

The disclosed technology is related to a system and a method for precise and programmable polynucleotide nicking based on a hybrid structure comprising one or more nicking molecules or chemical entities and a single-base-read nanopore sequencing device.

In the disclosed technology, one or more nicking molecules or chemical entities are grafted or conjugated in proximity to a nanopore of a real-time single-base-read nanopore sequencing device. To nick or cleave a polynucleotide molecule at a point immediately following, or a certain number of bases after, any desirable polynucleotide sequence, if the desirable polynucleotide sequence is detected by the single-base-read nanopore sequencing device, the nicking molecule or chemical entity may be activated by chemical, biological or physical means to nick or cleave the polynucleotide.

Examples of nicking molecules or chemical entities include, without limitation, ruthenium complexes with an extended π-system, which cleave DNA upon light irradiation (Sun, Y.; Joyce, L. E.; Dickson, N. M.; Turro, C. “Efficient DNA photocleavage by [Ru(bpy)₂(dppn)]²⁺, Chem. Comm. 46, 2426-2428, 2010); and coralyne, which can cause DNA single strand (s.s.) breaks upon photosensitization (Patro, B. S.; Bhattacharyya, R.; Gupta, P.; Bandyopadhyay, S. “Mechanism of coralyne-mediated DNA photo-nicking process”, J. Photochem. Photobiol. B, Biol. 194, 140-148, 2019).

Since the nicking or cleaving action is initiated only when a desirable polynucleotide sequence is read and confirmed in-situ by the single-base-read nanopore polynucleotide sequencing device performing real-time sequencing, the polynucleotide molecule can be nicked or cleaved at will.

A nick after another sequence on the same polynucleotide molecule can be achieved by monitoring the real-time sequencing result for said another sequence. Once the another sequence is read, nicking or cleaving is triggered. In this case, the system for precise and programmable polynucleotide nicking can achieve nicking or cleaving after a different sequence on the same polynucleotide molecule.

A system consisting of one or more nicking molecules coupled or conjugated to a real-time single-base-read polynucleotide sequencing device is provided. The real-time single-base-read polynucleotide sequencing device is one that can probe and identify individual bases in real-time as a polynucleotide molecule passes through a nanopore of the single-base-read polynucleotide sequencing device. The one or more nicking molecules can be activated by chemical, biological or physical excitation to nick the polynucleotide molecule instantaneously. A method to achieve precise and programmable nicking or cleaving of a polynucleotide is also provided based on this system.

The following examples illustrate the present teachings and are in no way limiting to the potential applications of the present subject matter. In the following detailed discussion, specific applications of DNA molecule as the polynucleotide molecule will be discussed, but one skilled in the art would understand that an RNA molecule or hybrid DNA-RNA molecule could similarly be used.

### Example 1: General Configuration

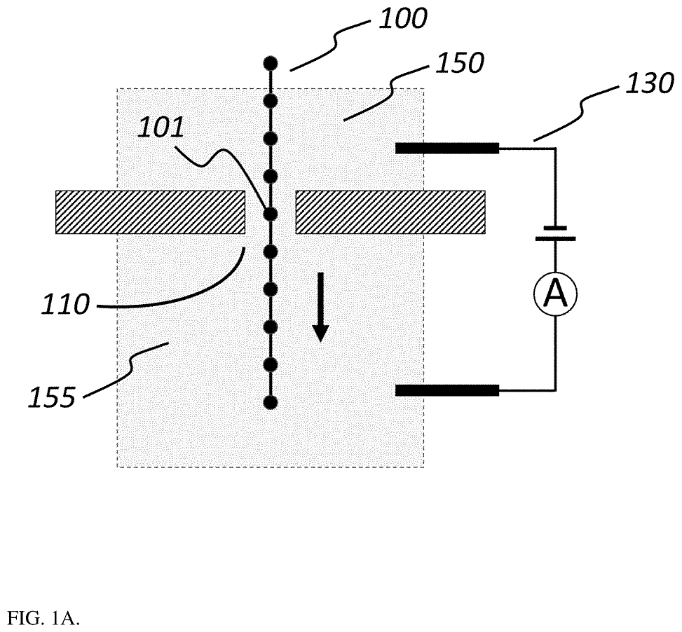

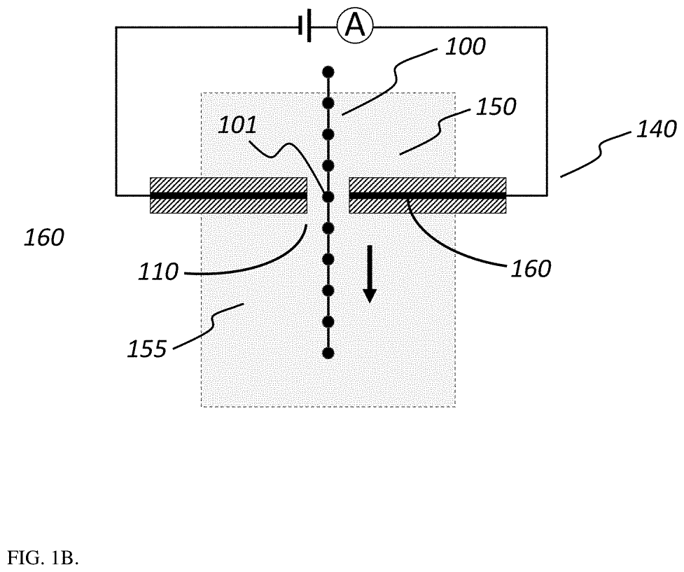

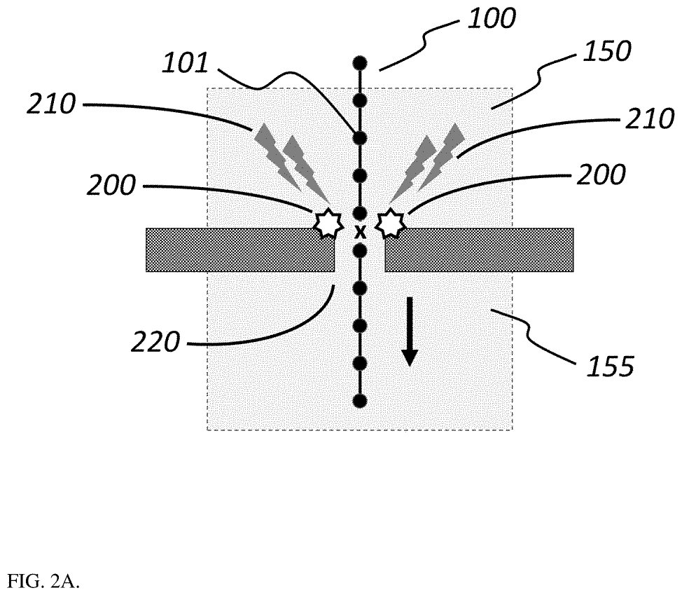

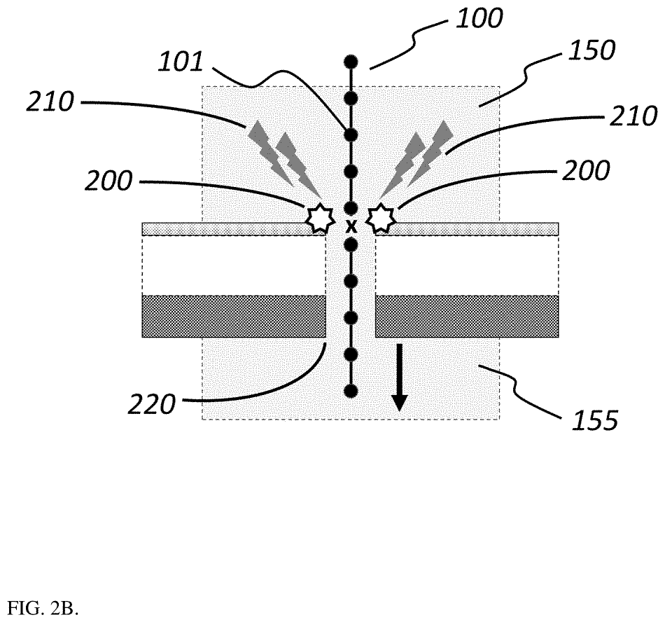

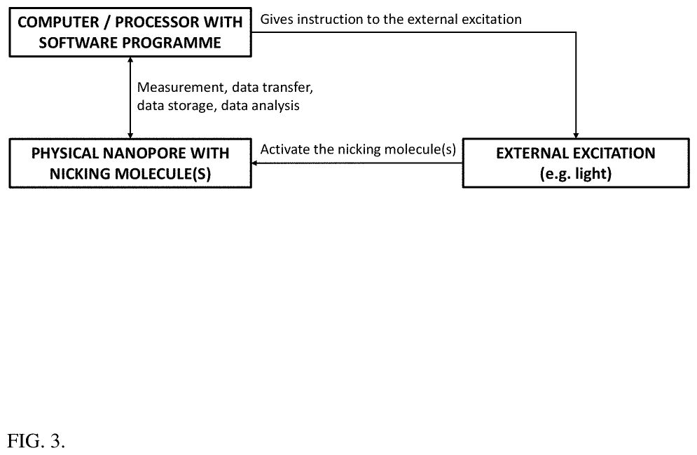

FIG. 1A is a schematic drawing showing an exemplary nanopore device for polynucleotide sequencing by ion blockage current measurement. FIG. 1B is a schematic drawing showing an exemplary nanopore device for polynucleotide sequencing by in-plane electrical signal measurement. FIG. 2A is a schematic drawing illustrating an exemplary setup for the disclosed technology comprising one or more nicking molecules grafted at an opening of a nanopore of a single-base-read nanopore sequencing system for precise and programmable polynucleotide nicking immediately after the desirable polynucleotide sequence is read. FIG. 2B is a schematic drawing illustrating an exemplary setup for the disclosed technology comprising one or more nicking molecules grafted at a predetermined distance above an opening of a nanopore of a single-base-read nanopore sequencing system for precise and programmable DNA nicking at a designated site away from the desirable DNA sequence read. The downward arrows in all FIGS. 1A, 1B, 2A and 2B, indicate the direction of DNA translocation. FIG. 3 is a flow chart illustrating the method of the disclosed technology.

A single-base-read nanopore polynucleotide sequencing device is a nanopore polynucleotide sequencing device that can achieve real-time single-base-read sequencing by probing an individual base 101 of a polynucleotide molecule 100 and measuring one or more properties of the individual base 101 by a direct or indirect method. The shape of the nanopore 110 can be different and should not be restricted to the cylindrical shape as shown in FIGS. 1-3, which are examples for illustrative purposes only.

Among approaches, electrical signal measurement is one of the most commonly adopted methods to probe bases by single-base-read nanopore polynucleotide sequencing devices. The electrical signal measurement can be achieved by, without limitation, ion blockade measurement (with electrode configuration 130 in FIG. 1A) or in-plane measurement (with electrode configuration 140 in FIG. 1B).

For ion blockade measurement, ion current is measured across the nanopore membrane separating two compartments (cis- 150 and trans- 155) as each of the individual bases along the polynucleotide molecule travel through the nanopore. For in-plane measurement, built-in or embedded electrodes 160 can be used to measure the in-plane electrical properties (e.g., tunneling current, resistance) as each of the individual bases along the polynucleotide molecule travel through the nanopore. The position of built-in or embedded electrodes can vary from the top to the bottom of the nanopore depending on design of device. Additional electrodes may be applied in the trans and cis compartments to drive the polynucleotide molecule to translocate through the nanopore. The single-base-read nanopore sequencing device should further comprise a computer or other processor to read the ion current measurement of the built-in or embedded electrodes in real time, store the measurements and convert the measurements to corresponding individual bases and a real time sequence of the polynucleotide molecule as it passes through the nanopore. The computer or other processor should store and access a target sequences or list of target sequences, for example, and continuously compare the real time determined sequence of the polynucleotide molecule with the target sequence. When a match is detected, the computer or other processor should transmit a signal that triggers an external excitation of the nicking or cleaving molecule of the present device. Those skilled in the art may use single-base-read nanopore sequencing devices other than those described here, but also suitable for the disclosed technology.

Nicking molecules 200 according to the disclosed technology are natural or synthetic chemical molecules that can perform a nicking or cleaving action on a DNA molecule when an external excitation (chemical, biological, physical, etc.) 210 is applied. They do not require any recognition domain for sequence recognition, sequence identification and/or binding, and their nicking or cleaving action is therefore not sequence-specific. Chemical excitations include, but are not limited to, those induced by a cation (e.g., H⁺, K⁺, Mg²⁺), anions (e.g., OH⁻, Cl⁻) and radicals (e.g., .OH, .O₂). Biological excitations include, but are not limited to, those induced by biological compounds and macromolecules, for instance, proteins, enzymes and RNAs. Physical excitations include, but are not limited to, thermal, light and electrical excitations.

As illustrated in FIGS. 2A-B, one or more nicking molecules 200 are grafted at the opening of a nanopore 220 in FIG. 2A (the nanopore 220 can be in a same single layer structure with the electrodes, as the nanopore 110 in FIG. 1A, or a multilayer structure with the electrodes, as nanopore 110 in FIG. 1B) for precise and programmable DNA nicking immediately after the desirable DNA sequence is read, or grafted at a predetermined distance above the opening of a nanopore 220 in FIG. 2B (the nanopore 220 can be in a single layer structure or multilayer structure with the electrodes, as nanopore 110 in FIGS. 1A-B, respectively) for precise and programmable DNA nicking at a designated site away from the desirable DNA sequence read. Other configurations involving, but not limited to, the relative position and number of components, would be apparent to those skilled in the art according to the embodiments described herein. Variation and modification of the exact configuration will also be apparent to those skilled in the art so long as the spirit of the disclosed technology is maintained.

In one embodiment, nicking molecules can be grafted to a nanopore as follows. A designated nanoscale region (e.g., a nanodot or nanopatch) located near an edge of a mouth of the nanopore (i.e., an opening of the nanopore in plane with either the cis or trans side of the membrane or substrate in which the nanopore is situated), and in a particular embodiment, directly adjacent to an edge of the mouth of the nanopore, is firstly deposited with a metal (including, without limitation, gold (Au), silver (Ag) and copper (Cu)) by vacuum deposition techniques, such as focused ion beam and electron-beam-induced deposition as described in Dhawan, A.; Gerhold, M.; Russell, P.; Vo-Dinh, T.; Leonard, D. “Fabrication of metallic nanodot structures using focused ion beam (FIB) and electron-beam-induced deposition for plasmonic waveguides”, Proc. of SPIE, 7224, 722414, 2009, and Shimojo, M.; Zhang, W.; Takeguchi, M.; Tanaka, M.; Mitsuishi, K.; Furuya, K. “Nanodot and nanorod formation in electron-beam induced deposition using iron carbonyl”, Jpn. J. Appl. Phys. 44(7B), 5651, 2005. In an exemplary implementation of the present embodiment, a gold nanodot may be used. In a particular implementation of the present embodiment, coralyne is chosen as an example of light-responding DNA nicking molecule (Patro, B. S.; Bhattacharyya, R.; Gupta, P.; Bandyopadhyay, S. “Mechanism of coralyne-mediated DNA photo-nicking process”, J. Photochem. Photobiol. B, Biol. 194, 140-148, 2019). Coralyne can perform a nicking action on a DNA molecule upon light irradiation. To graft a coralyne molecule to the nanopore, a methyl group (—CH₃) in any methoxy (—OCH₃) branch of the coralyne molecule, or a hydrogen group (—H) in any methyl group (—CH₃) of the coralyne molecule may be substituted by a sulphide group (—SH) using any known or suitable synthetic chemistry technique, resulting in an S-modified coralyne. The S-modified coralyne can be attached to the Au nanodot formed adjacent to the nanopore through spontaneous formation of an Au—S linkage by any suitable means, such as contact with an appropriate solvent (e.g., ethanol). See, for example, Inkpen, M. S., Liu, Z. F., Li, H., Campos, L. M., Neaton, J. B., & Venkataraman, L. (2019). Non-chemisorbed gold-sulfur binding prevails in self-assembled monolayers. Nature chemistry, 11(4), 351-358. The coralyne molecule is thus linked to the Au nanodot adjacent to the mouth of the nanopore. One skilled in the art would understand that Au and S-modified coralyne are among the many possible routes to grafting a nicking molecule to a nanopore. Any suitable conjugation or grafting technique other than those described here, but also suitable for the disclosed technology, may be used.

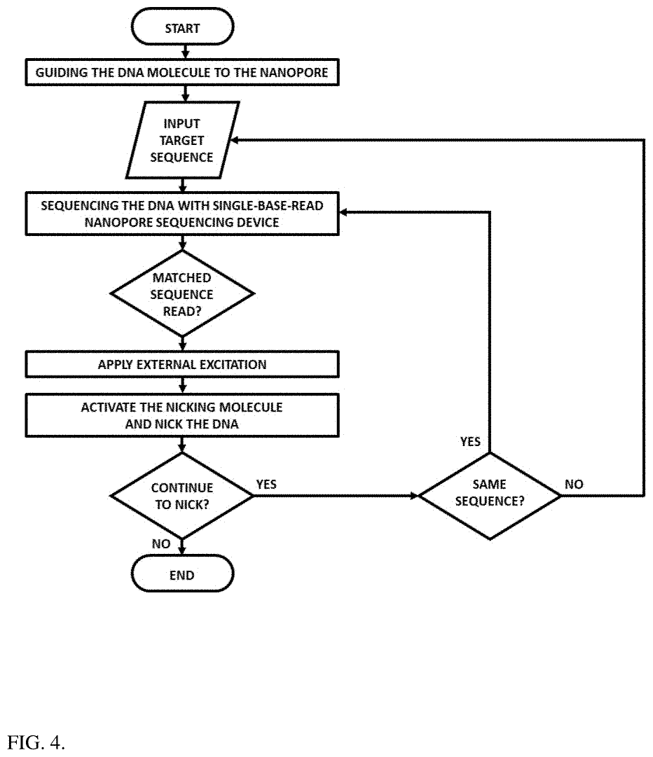

When a DNA molecule 100 is guided to translocate through the nanopore of the single-base-read sequencing device, such as those exemplified in FIGS. 2A-B, the single-base-read sequencing device continuously and in real time reads each individual base of the DNA molecule and thereby sequences the DNA molecule in real-time. The sequence data are continuously recorded and stored by a computer or processor using a software program. The software program implemented on the computer or processor can also continuously compare the read sequence with a predetermined target sequence assigned by the operator. If it is desired to create a nick along the DNA molecule after a predetermined target sequence, for example ATCGAC, once the single-base-read sequence device reads ATCGAC, as determined by the software program implemented by the computer or processor, the software program will generate instructions that will be transmitted by the computer or processor to an external excitation source to generate an external excitation 210 (e.g., to a light source to generate light that triggers nicking action of coralyne) so as to activate the one or more nicking molecules 200 (e.g. coralyne), which nick the DNA to create a nick, as indicated by “x” in FIG. 2A and FIG. 2B. Depending on the relative position of the nicking molecules with respect to the nanopore of the single-base-read sequencing device, the nick created can be immediately after, or some length after, the sequence ATCGAC. After nicking, the external excitation is stopped and sequencing resumes as instructed by the software program. (FIG. 3). The process can be repeated when further nicks after ATCGAC are required. If nicks are required after another target sequence, for example GTACAG, the process continues in the same fashion except that only when GTACAG is read (instead of ATCGAC previously targeted) is the external excitation applied and the nicking triggered. Nicking can be repeated after the same sequence or different sequence along the same DNA with the same system. The whole process is summarized by the flow chart in FIG. 4.

As the disclosed technology does not rely on any sequence-specific recognition domain, nicks can be created after any target DNA sequence of unrestricted length.

In another instance, a combination of prior recognition and distance can be defined a priori to activate nicking. Since there may be a “dead space” in between ends of each recognition site (position), to get rid of this dead space or nick prior to or in a recognition site, the one or more nicking molecules may be grafted or coupled to the trans side of the nanopore. Otherwise, if the one or more nicking molecules were grafted or coupled to the cis side of the nanopore, reversing the translocating direction of the DNA molecule after detecting the recognition sequence may allow for nicking prior to or in the recognition site. An example of reversing the translocating direction is described in Gershow, M.; Golovchenko, J. A. “Recapturing and Trapping Single Molecules with a Solid-State Nanopore”, Nat. Nanotech. 2, 775-779, 2007.

### Application Examples

The disclosed system and method decouple naturally and enzymatically nicking actions that require coordinated efforts of a protein recognition domain and a protein catalytic domain, and replaces these two usually inseparable actions with two separate controllable and programmable elements, namely a nanopore sequencer of real-time single base resolution and a nicking components of the precise and programmable nicking molecule. This disclosed technology will enable many transformative applications. The following are two examples.

### Example 1: DNA Based Information Storage

A novel DNA punch card with selectable or designable DNA substrates, and variable programmable nicking sites (rather than a set of predetermined recognition sites on a known DNA substrate, e.g., fokI sites on lambda phage DNA): the disclosed technology affords potentially higher density of “punch holes” or “registers” on the same unit length of DNA substrate than existing technology.

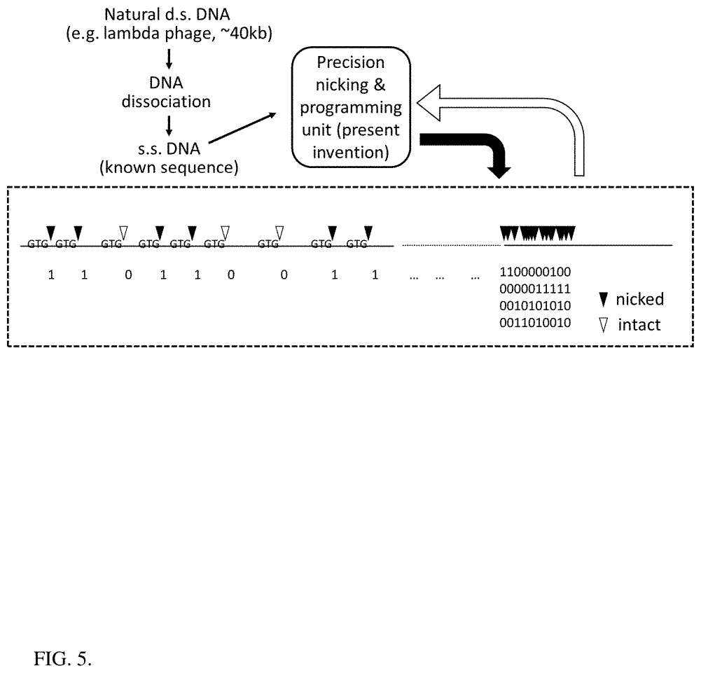

FIG. 5 is a schematic diagram illustrating a random access memory employing a nucleic acid as an example of an encoding scheme. In an embodiment, the nucleic acid may be a DNA molecule.

This example denotes a potential application of the disclosed technique in which known natural DNA molecules are used as the writing material to enact random access memory.

A double-stranded DNA (d.s. DNA) molecule, naturally occurring or synthesized, may be dissociated into single stranded DNA (s.s. DNA) molecules, a top strand and a bottom strand (the dissociation can be performed separately, or in a device such as, without limitation, a microfluidic flow cell, which may be directly connected with the single-base-read sequencing device), before translocating the top strand and the bottom strand through the nanopore of the precise and programmable nicking single-unit (i.e., the single-base-read nanopore sequencing device with the one or more nicking molecules, hereafter “single unit”) of the disclosed technology can be programed to nick both the top and bottom strands of the original DNA molecule in concert, so that when rehybridized after a series of programmatic nicking actions on the top and bottom strands, a DNA molecule with binary codes as information storage is generated.

The solid curved arrow in FIG. 5 denotes binary encoding via precise and selective nicking as the s.s. DNA molecule translocates through the single unit. The open curved arrow denotes reading the unique sequences of the s.s. DNA fragments generated by the nicking by the same single unit when the translocation direction is reversed.

The triplet sequence “GTG” in FIG. 5 is selected as an illustrative nicking recognition sequence; on average, there would be about a single site every 64 nucleotides. Each site can store one bit of information; the presence of nick at the recognition site represents “1”, and the absence “0”. Each string of ten sites can be a “register” of about 640 nucleotides long of the known reference DNA sequence used. For example, if a lambda phage genome (about 48 kb in size) is used as the reference DNA molecule, then there are about over 64 registers possible. A larger reference DNA selected from a natural genome would have a larger number of registers. For example, a genome of E. coli of about 5 Mb in size would have proportionally ten times more registers.

While the writing process during encoding is sequential (as the single DNA molecule is translocated through the single unit), the reading process can be conveniently performed at random, as the encoding fragments would have their own unique sequence that can be aligned with the whole genome reference sequence, allowing for precisely and accurately specifying the nicked sites. Solid arrow heads and open arrow heads respectively denote the presence and absence of nicks at the selected sites.

### Example 2: Synthetic Biology—Use of Native Genomic DNA Molecules as Starting Material to Systematically Build Artificial-Alternative Splicing of Eukaryotic Genomes

Synthetic biology has emerged as a promising field for studying a wide range of biological phenomena and for creating unprecedented artificial systems for new uses.

Native biological systems are regarded as results of natural selection, and presumably near optimal solutions with respect to the evolution history experienced by the native biological systems.

Synthetic biology holds the power to systematically “rework” any existing biological systems by means of chemical synthesis of an entire genome or of the complete genetic representation of a biological pathway, or merely of an artificial new member to be added to an existing superfamily of proteins. This reworking is achieved using purposefully altered or synthetic genomes designed to by-pass the limitations of natural selection, for examples, a totally chemical synthesized bacterial genome or a “v 2.0” yeast chromosome. Examples of such totally chemical synthesized genomes and chromosomes are described in, for example, Gibson, D. G.; Glass, J. I.; Lartigue, C.; Noskov, V. N.; Chuang, R. Y.; Algire, M. A.; Benders, G. A.; Montague, M. G.; Ma, L.; Moodie, M. M.; Merryman, C.; Vashee, S.; Krishnakumar, R.; Assad-Garcia, N.; Andrews-Pfannkoch, C.; Denisova, E. A.; Young, L.; Qi, Z. Q.; Segall-Shapiro, T. H.; Calvey, C. H.; Parmar, P. P.; Hutchison, C. A.; Smith, H. O.; Venter, J. C. “Creation of a bacterial cell controlled by a chemically synthesized genome”. Science 329, 52-56, 2010 and in Dymond, J. S.; Richardson, S. M.; Coombes, C. E.; Babatz, T.; Muller, H.; Annaluru, N.; Blake, W. J.; Schwerzmann, J. W.; Dai, J.; Lindstrom, D. L.; Boeke, A. C.' Gottschling, D. E.; Chandrasegaran, S.; Bader, J. S.; Boeke, J. D., “Synthetic chromosome arms function in yeast and generate phenotypic diversity by design”, Nature 477, 471-476, 2011. While these examples are proof-of-principle, they are prohibitively costly in resources and effort.

Moreover, chemical DNA synthesis (using phosphonamidite chemistry) suffers from inherent inaccuracy and particularly cost-effective high-throughput synthesis, e.g., microarray based light-directed synthesis can harbor a high error rate. Therefore, expensive and time-consuming error correction by means of site-specific mutagenesis or other demanding methods are necessary to eliminate the random errors in these synthetic DNA molecules to ensure integrity of the artificial genes. Examples of site-specific mutagenesis or other demanding methods can be found in Wan, W.; Li, L.; Xu, Q.; Wang, Z.; Yao, Y.; Wang, R.; Zhang, J.; Liu, H.; Gao, X.; Hong, J. “Error removal in microchip-synthesized DNA using immobilized MutS”, Nucleic Acids Res. 42 (12), e102, 2014, and in Ner, S. S.; Smith, M. “Role of intron splicing in the function of the MATa1 gene of Saccharomyces cerevisiae”. Mol. Cell Biol. 9 (11), 4613-20, 1989.

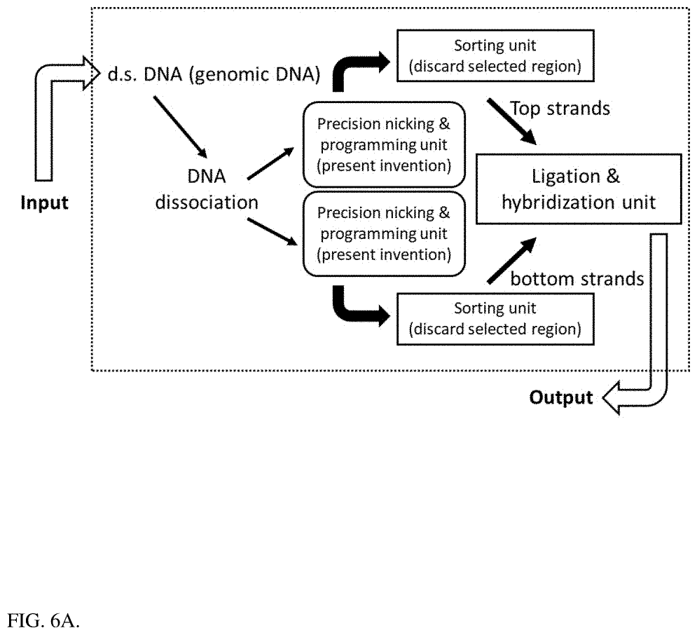

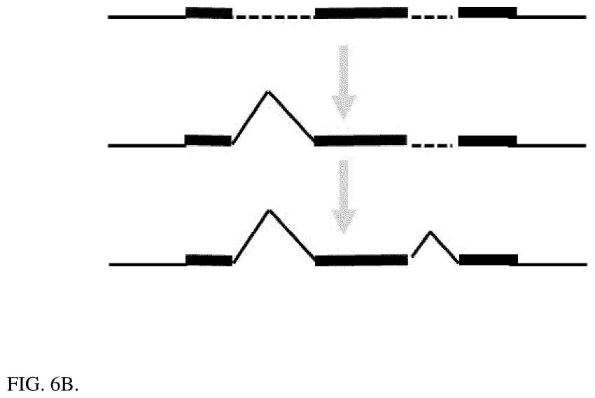

The disclosed technology allows for building accurate synthetic systems cost-effectively with native genomic DNA molecules, or other easily accessible or designable nucleic acid molecules, as the starting materials. A schematic for a method of synthesizing an intron-less yeast genome—i.e., removal of the two native introns from the yeast MATa1 gene—illustrating this process is presented in FIGS. 6A and 6B. FIG. 6A is a flow diagram showing an example of application in synthetic biology using natural DNA instead of conventionally chemical synthesized DNA oligonucleotides. FIG. 6B is a diagram showing an example of a sequence of removal of two introns from the yeast MATa1 genomic DNA (in two iterations of single intron removal) using an embodiment of the disclosed technology.

The block diagram of FIG. 6A represents the basic units in a double-stranded DNA manipulation system, in which selective regions can be read and removed from top and bottom strands, and then remaining regions are ligated back and hybridized to generate the newly engineered DNA molecule.

Such output can be used as the starting materials or input of the next round of DNA molecule manipulation; i.e., in an iterative fashion, to eventually obtain the final designed products, which in turn can be used to build synthetic biological systems; e.g., yeast MATa1 genes without its two native introns, as shown in FIG. 6B. An example of such DNA molecule manipulation is described in Kessler, C.; Manta, V. “Specificity of restriction endonucleases and DNA modification methyltransferases a review”. Gene 92 (1-2), 1-248, 1990.

It is therefore possible to incorporate heterogeneous and chemically synthesized gene cassettes by introducing oligonucleotides into the ligation & hybridization unit of this manipulation system to enhance the artificial aspects of desired synthetic systems.

The foregoing description is illustrative of particular embodiments, but it is not meant to be a limitation upon the practice thereof. The following claims, including all equivalents thereof, are intended to define the scope of the disclosed technology.

It should be understood that many additional changes in the details, materials, steps and arrangement of parts, which have been herein described and illustrated to explain the nature of the subject matter, may be made by those skilled in the art within the principle and scope of the invention as expressed in the appended claims.

What is claimed is:

1. A system for precise and programmable polynucleotide nicking, comprising:
one or more nicking molecules to perform a nicking or cleavage action on a polynucleotide molecule;
a single-base-read nanopore sequencing device that is configured to achieve real-time single-base-read sequencing of the polynucleotide molecule by probing a property of an individual base of the polynucleotide molecule in a nanopore of the single-base-read nanopore sequencing device, acquiring a measurement of the probed property of the individual base of the polynucleotide molecule, transmitting the measurement to a processor configured to convert the measurement to a base identity representing the individual base and sequentially store the base identity with base identities of previously measured individual bases of the polynucleotide molecule as polynucleotide molecule sequence data;
a processor configured to read the polynucleotide molecule sequence data and recognize a predetermined sequence; and
a transmitter to transmit a signal to produce an excitation of the one or more nicking molecules that triggers the nicking or cleavage action on the polynucleotide molecule.

2. The system of claim 1, wherein
the predetermined sequence is any sequence of base identities and the nicking or cleaving action on the polynucleotide molecule occurs without binding by the one or more nicking molecules to a recognition site of the polynucleotide molecule.

3. The system of claim 1, wherein
the one or more nicking molecules is grafted at a known distance to the nanopore of the real-time single-base-read nanopore sequencing device.

4. The system of claim 1, wherein
each of the one or more nicking molecules is either a natural or synthetic molecule, and
the nanopore is a biological nanopore, an inorganic nanopore, or an organic nanopore.

5. The system of claim 1, wherein
the external excitation may be a chemical, biological or physical excitation.

6. The system of claim 1, wherein
the real-time single-base-read nanopore sequencing device probes and identifies an individual base of a polynucleotide molecule in a real-time fashion when the polynucleotide molecule translocates through the nanopore.

7. The system of claim 6, wherein
the real-time single-base-read nanopore sequencing device is programmed to read and identify at least one predetermined sequence of individual bases of the polynucleotide molecule.

8. The system of claim 1, wherein
the nanopore has a shape that allows translocation of a polynucleotide molecule.

9. The system of claim 7, wherein
the property is an ion blockage current of the individual base in the nanopore.

10. A method for polynucleotide nicking, comprising:
guiding a polynucleotide molecule to the nanopore of the real-time single-base-read nanopore sequencing device;
guiding the polynucleotide molecule to enter and translocate through the nanopore;
determining a target sequence for a nick or cleave;
sequencing the polynucleotide by the real-time single-base-read nanopore sequencing device;
after reading the target sequence by the real-time single-base-read nanopore sequencing device, applying an external excitation to trigger one or more nicking molecules, and thereby nicking or cleaving an adjacent position of the polynucleotide molecule;
in the case of a requirement to further nick or cleave the same target sequence, continuing the sequencing, reading and nicking or cleaving steps;
in the case of a requirement to nick or cleave another target sequence, determining said another target sequence as a new target sequence;
after reading the new target sequence by the real-time single-base-read nanopore sequencing device, applying the external excitation to trigger the one or more nicking molecules to nick the adjacent position of the polynucleotide molecule; and
repeating the process until completion of all nicks or cleaves.

11. The method of claim 10, further comprising:
controlling the guiding of a polynucleotide molecule to the nanopore of the real-time single-base-read nanopore sequencing device by microfluidic techniques or by electrokinetic techniques.

12. The method of claim 10, further comprising:
selecting a predetermined sequence of bases as a nicking or cleaving recognition sequence;
sequencing of the polynucleotide molecule in real-time;
to write a register “1”, generating a nick after reading the nicking or cleaving recognition sequence by triggering external excitation to activate the one or more nicking or cleaving molecules;
to write a register “0”, skipping a nicking or cleaving action even after reading the nicking or cleaving recognition sequence;
continuing the process until the desired sequence of registers is made.

13. A method of achieving polynucleotide based information storage through generation of nicks or cleavings as registers on a polynucleotide molecule in one or more nicking molecules, comprising:
predetermining a predetermined sequence of bases as a nicking or cleaving recognition sequence;
sequencing of the polynucleotide molecule in real-time;
to write a register “1”, generating a nick after reading the nicking or cleaving recognition sequence by triggering external excitation to activate the one or more nicking or cleaving molecules;
to write a register “0”, skipping a nicking or cleaving action even after reading the nicking or cleaving recognition sequence;
continuing the process until the desired sequence of registers is made.

14. The method of claim 13, wherein the nicking or cleaving recognition sequence comprises at least two bases.

15. The method of claim 13, comprising selecting a nick register immediately after or a chosen number of bases after the nicking or cleaving recognition sequence.

16. A method to modify a polynucleotide molecule using the system of claim 1, wherein the polynucleotide molecule is a double stranded DNA molecule, the method comprising steps of:
predetermining one or more regions of the double stranded DNA molecule to remove;
programming the system to recognize target sequences to trigger nicking by the system at either end of the one or more regions
denaturing the double stranded DNA molecule into a top strand and a bottom strand;
sequencing the top strand and bottom strand in real-time using the system;
reading the one or more selected regions and removing the one or more regions from the top and bottom strands in real-time using the system; and
ligating the desired remaining regions to form modified top and bottom strands and hybridizing the ligated top and bottom strands to generate a modified double stranded DNA molecule.

17. The method of claim 16, further comprising:
using the modified DNA molecule as the double stranded DNA molecule to be modified in a next round modification in an iterative fashion.
